(19) United States
(12) Patent Application Publication
(10) Pub. No.: US 2006/0127272 A1
(43) Pub. Date: Jun. 15, 2006

(54) Stabilized aqueous colorant preparations

(76) Inventors: Klaus Saitmacher, Kriftel (DE); Josef Geisenberger, Sulzbach (DE); Karl-Heinz Schweikart, Eschborn (DE); Till Borchert, Langen (DE)

Correspondence Address: CLARIANT CORPORATION;INTELLECTUAL PROPERTY DEPARTMENT, 4000 MONROE ROAD, CHARLOTTE, NC, 28205, US

(21) Appl. No.: 10/560,091

(22) PCT Filed: May 21, 2004
(86) PCT No.: PCT/EP04/05458

§ 371 Date: Dec. 9, 2005

(30) Foreign Application Priority Data
Jun. 11, 2003 (DE) 10326210.5

(51) Int. Cl. A61L 2/16

(52) U.S. Cl. 422/28

(57) ABSTRACT

The invention relates to the use of an antimicrobially effective amount of a mixture from 5-chloro-2-methyl-4-isothlazolin-3-one, 2-methyl-4-isothiazolin-3-one and 2-methyl-4-isothiazolin-3-one and 2-bromo-2-nitropropane-1,3-diol as a biocide in aqueous colorant preparations that have a heavy metal ion concentration of greater or equal 20 ppm. The invention effectively prevents the formation of precipitations.

This invention relates to the field of colorants, in particular for use in recording fluids for writing and recording implements, for example for ink jet processes.

The ink jet process is a non-impact printing process wherein droplets of a recording fluid are directed from one or more nozzles onto the substrate to be printed. To obtain prints of high sharpness and good resolution, a recording fluid and the colorants it contains have to meet high requirements, in particular with regard to purity, particle absence, solubility, stability in storage, viscosity, surface tension and also conductivity.

The dyes used in the inks are key in this. Although a large number of dyes have been developed, there are only a few which meet the requirements of a state of the art ink jet printing operation. Heavy metal complex colorants play an important part, in particular copper complex dyes and pigments. As well as dye-based inks, pigmented inks have recently come to be increasingly used in ink jet printing.

It is known to admix aqueous colorant preparations and inks, for example ink jet inks, with biocidal actives to control growth of undesirable microorganisms such as bacteria and molds, yeasts and slimes.

DE-A-101 12 367 discloses using a synergistic mixture of methyl-2H-isothiazol-3-one and 2-bromonitropropanediol to control microbial growth in industrial materials.

JP 2000 226545-A proposes a series of further biocides for use in ink jet inks.

It has emerged, however, that colorants comprising complex-bound heavy metal ions or colorants which are notionally heavy metal free but contaminated by heavy metal ions may give rise to precipitations due to reaction of the heavy metal ion with customary biocides or customary biocide mixtures whereby nobzzles in ink jet print heads in particular may become clogged. Such heavy metal ions, in particular multiply charged metal cations such as, for example, Cu²⁺, Fe³⁺ or Al³⁺, may be present as a residual impurity in the dye solution, in the pigment powder or in the pigment presscake and transported into the ink via the dispersing operation. Moreover, there may be heavy metal impurities in dispersants or other ink additives used. Since the abovementioned biocide-heavy metal complexes often also have distinct intrinsic colorations, this can have adverse repercussions for the color properties of the inks and hence of the ink jet prints produced. As well as printer nozzle clogging, insoluble complexes. may kogate on the heating elements of the printer or lead to problems in relation to filtering operations.

The present invention therefore has for its object to provide an effective biocidal composition for aqueous heavy-metal-containing colorant preparations and inks which does not evoke any precipitations whatsoever. Furthermore, the biocidal composition shall protect the colorant preparations and inks in question from germs over a wide pH range, in particular between pH 3 and 10, since such colorant preparations are often shipped in an acidic or alkaline medium for stability reasons.

We have found that these objects are achieved, surprisingly, by a mixture of 5-chloro-2-methyl-4-isothiazolin-3-one (CM IT), 2-methyl-4-isothiazolin-3-one (MIT) and 2-bromo-2-nitropropane-1,3-diol (bronopol).

The present invention accordingly provides for the use of an antimicrobially effective amount of a mixture of 5-chloro-2-methyl-4-isothiazolin-3-one, 2-methyl-4-isothiazolin-3-one and 2-bromo-2-nitropropane-1,3-diol as a biocide in aqueous colorant preparations, preferably aqueous recording fluids, in particular ink jet inks, having a heavy metal ion concentration of not less than 20 ppm and in particular not less than 50 ppm.

The upper limit of the heavy metal ion concentration should not be above the maximum concentration of the biocidal mixture, of course. Heavy metal precipitates are still effectively prevented at metal concentrations of about 500 ppm without the amount of the biocidal mixture having to be raised beyond the economically acceptable.

The antimicrobial mixture can of course also be used in aqueous colorant preparations where the problem of heavy metal precipitation does not arise at all or only to a small extent, for example at heavy metal ion concentrations of below 20 ppm.

The antimicrobially effective amount for the biocidal mixture has been determined to be a level of 0.001% to 0.1% by weight and preferably 0.01% to 0.05% by weight, reckoned as solid and based on the total weight of the aqueous colorant preparation.

The mixing ratios of the individual CMIT:MIT:bronopol constituents is preferably (0.005 to 0.1):(0.005 to 0.05):1 and in particular (0.03 to 0.065):(0.01 to 0.025):1. To simplify metering, the antimicrobial mixture is usually employed in the form of a dilute, for example 5% to 15% by weight, aqueous solution which may further comprise small amounts of inorganic salts, examples being magnesium nitrate and/or magnesium chloride. The amount in which this solution is used in the colorant preparation is then preferably in the range from 0.01% to 1% by weight and in particular in the range from 0.1% to 0.2% by weight, based on the total weight of the colorant preparation.

Of particular interest for the purposes of the present invention are colorants comprising complexed heavy metals.

Usable heavy metal complex colorants include reactive dyes, direct dyes, acid dyes, disperse dyes and pigments, each comprising in particular a complexed heavy metal from the group consisting of Cu, Co, Ni, Fe, Cr and Al. Copper complex dyes and pigments are particularly preferred.

Examples of such reactive dyes are C.I. Reactive Black 8, C.I. Reactive Black 31; C.I. Reactive Blue 7, C.I. Reactive Blue 14, C.I. Reactive Blue 21, C.I. Reactive Blue 28, C.I. Reactive Blue 38, C.I. Reactive Blue 82, C.I. Reactive Blue 89, C.I. Reactive Blue 158, C.I. Reactive Blue 182, C.I. Reactive Blue 190, C.I. Reactive Blue 203, C.I. Reactive Blue 216, C.I. Reactive Blue 220, C.I. Reactive Blue 244; C.I. Reactive Violet 1, C.I. Reactive Violet 5; C.I. Reactive Red 6, C.I. Reactive Red 23 and C.I. Reactive Brown 18.

Examples of direct dyes are C.I. Direct Blue 76, C.I. Direct Blue 84, C.I. Direct Blue 86, C.I. Direct Blue 87, C.I. Direct Blue 98, C.I. Direct Blue 199, C.I. Direct Blue 202, C.I. Direct Blue 290; C.I. Direct Black 112; C.I. Direct Brown 95 and C.I. Direct Violet 47.

Examples of acid dyes are C.I. Acid Blue 87, C.I. Acid Blue 185 and C.I. Acid Blue 249.

Examples of heavy metal complex pigments are C.I. Pigment Blue 15:1-15:4, C.I. Pigment Blue 17, C.I. Pigment Green 7, C.I. Pigment Green 37, C.I. Pigment Red 257, C.I. Pigment Red 271, C.I. Pigment Orange 65, C.I. Pigment Orange 68, C.I. Pigment Yellow 117, C.I. Pigment Yellow 129 and C.I. Pigment Yellow 153.

Particular preference for the purposes of the present invention is given to C.I. Direct Blue 199 (5a) (Copper complex), C.I. Reactive Black 31 (5b) (Copper complex), C.I. Reactive Black 8 (Copper/cobalt complex) and C.I. Reactive Red 23 (Copper complex).

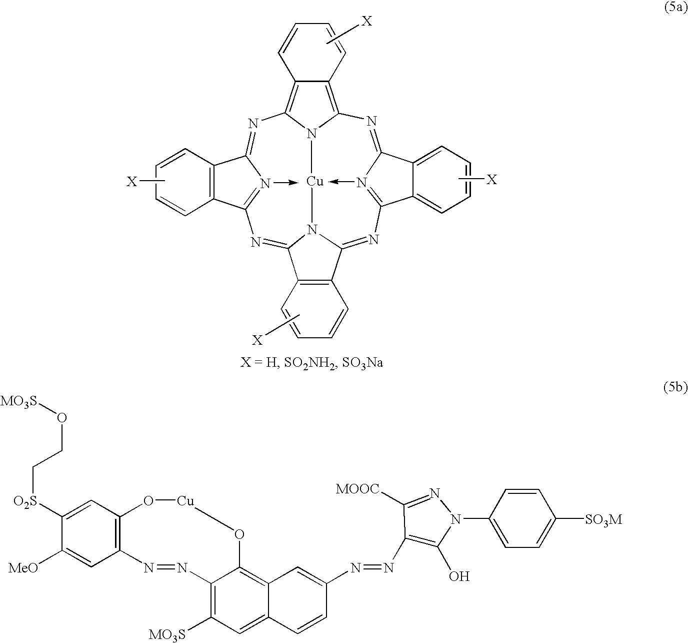

Further examples of copper complex dyes are compounds of the following formulae (5c) to (5l):

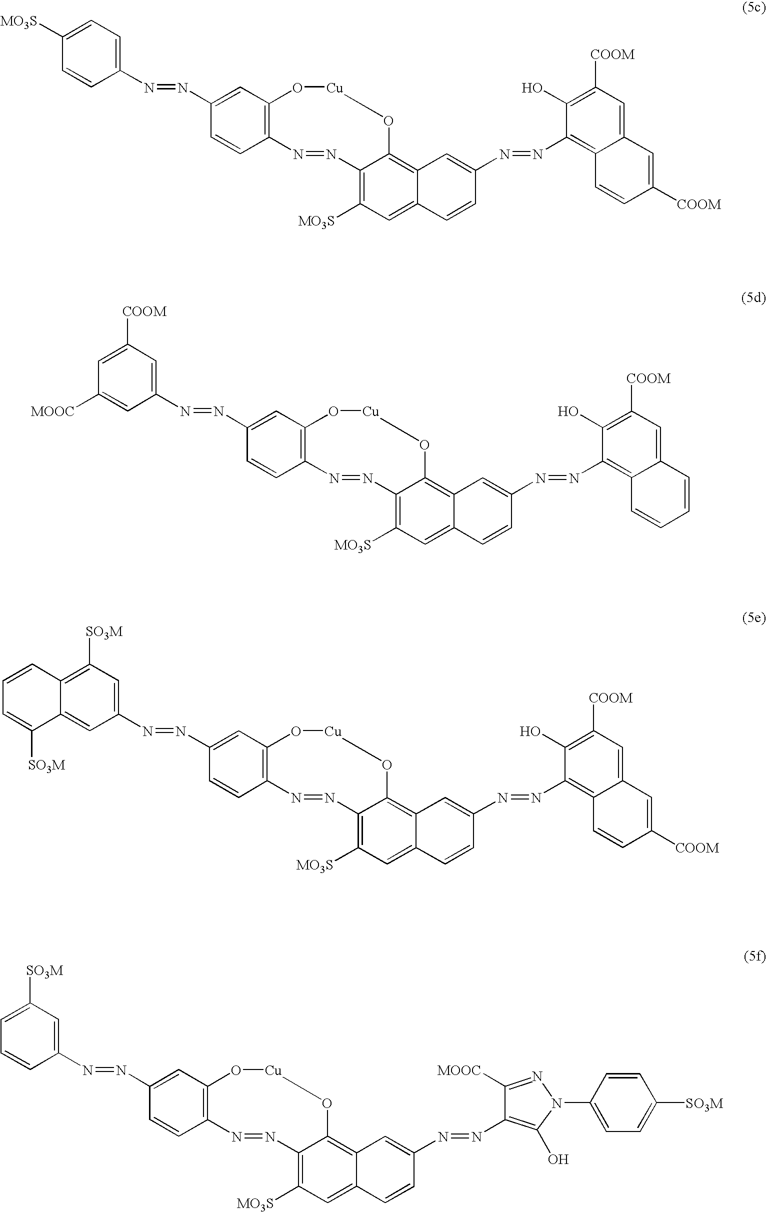

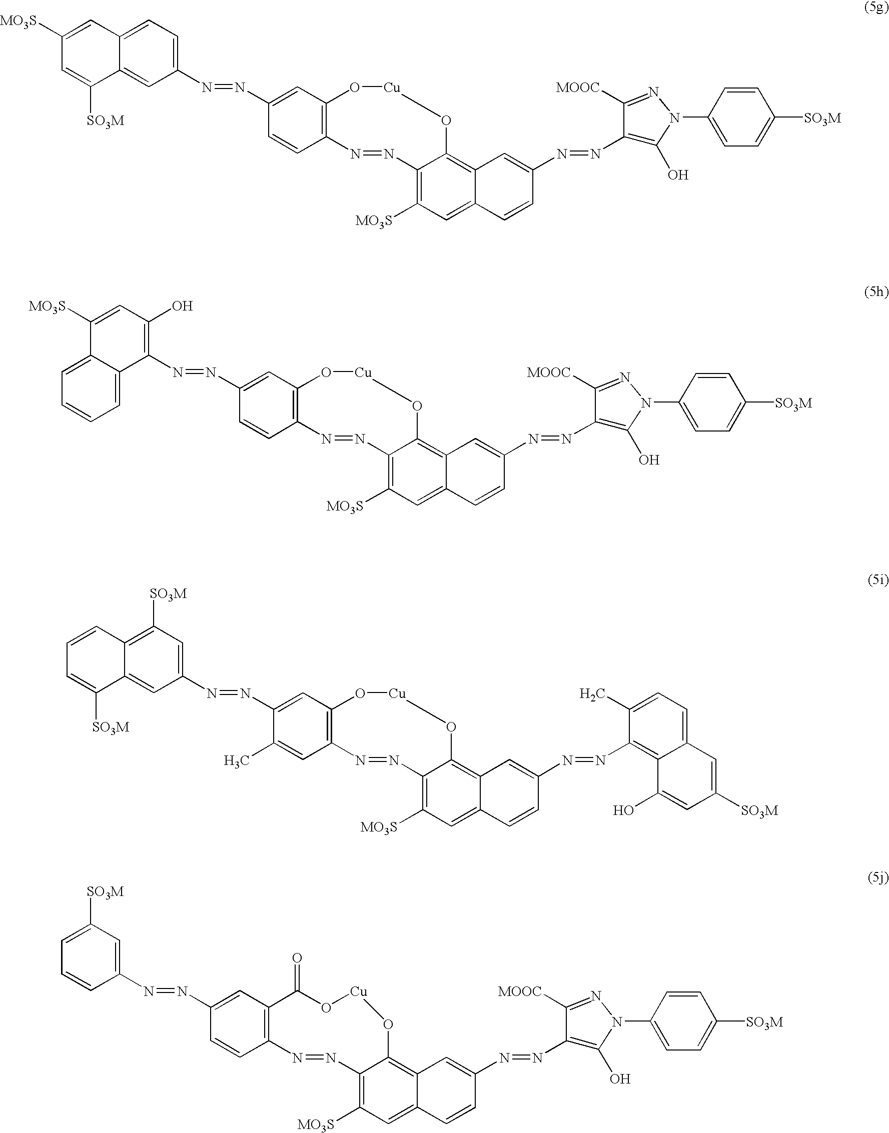

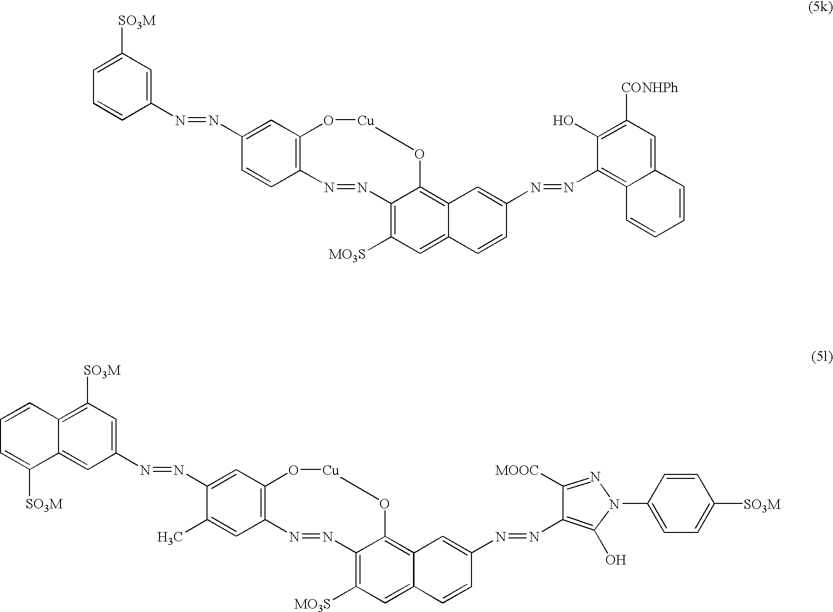

In the dyes of the formulae (5b) to (5l), M is preferably hydrogen and/or sodium, depending on the pH.

Useful organic pigments can be contaminated with heavy metal ions include monoazo, disazo, laked azo, β-naphthol, Naphthol AS, benzimidazolone, condensed disazo, azo metal complex pigments and polycyclic pigments such as for example phthalocyanine, quinacridone, perylene, perinone, thioindigo, anthanthrone, anthraquinone, flavanthrone, indanthrone, isoviolanthrone, pyranthrone, dioxazine, quinophthalone, isoindolinone, isoindoline and diketopyrrolopyrrole pigments or carbon blacks.

Useful inorganic pigments that can be contaminated with heavy metal ions include for example titanium dioxides, zinc sulfides, iron oxides, chromium oxides, ultramarine, nickel or chromium antimony titanium oxides, cobalt oxides and also bismuth vanadates.

An illustrative selection of particularly preferred organic pigments are carbon black pigments, for example lampblacks, or furnace blacks; monoazo and disazo pigments, in particular the Colour Index pigments Pigment Yellow 1, Pigment Yellow 3, Pigment Yellow 12, Pigment Yellow 13, Pigment Yellow 14, Pigment Yellow 16, Pigment Yellow 17, Pigment Yellow 73, Pigment Yellow 74, Pigment Yellow 81, Pigment Yellow 83, Pigment Yellow 87, Pigment Yellow 97, Pigment Yellow 111, Pigment Yellow 126, Pigment Yellow 127, Pigment Yellow 128, Pigment Yellow 155, Pigment Yellow 174, Pigment Yellow 176, Pigment Yellow 191, Pigment Red 38, Pigment Red 144, Pigment Red 214, Pigment Red 242, Pigment Red 262, Pigment Red 266, Pigment Red 269, Pigment Red 274, Pigment Orange 13, Pigment Orange 34 or Pigment Brown 41; β-naphthol and Naphthol AS pigments, in particular the Colour Index pigments Pigment Red 2, Pigment Red 3, Pigment Red 4, Pigment Red 5, Pigment Red 9, Pigment Red 12, Pigment Red 14, Pigment Red 53:1, Pigment Red 112, Pigment Red 146, Pigment Red 147, Pigment Red 170, Pigment Red 184, Pigment Red 187, Pigment Red 188, Pigment Red 210, Pigment Red 247, Pigment Red 253, Pigment Red 256, Pigment Orange 5, Pigment Orange 38 or Pigment Brown 1; laked azo and metal complex pigments, in particular the Colour Index pigments Pigment Red 48:2, Pigment Red 48:3, Pigment Red 48:4, Pigment Red 57:1, Pigment Red 257, Pigment Orange 68 or Pigment Orange 70; benzimidazoline pigments, in particular the Colour Index pigments Pigment Yellow 120, Pigment Yellow 151, Pigment Yellow 154, Pigment Yellow 175, Pigment Yellow 180, Pigment Yellow 181, Pigment Yellow 194, Pigment Red 175, Pigment Red 176, Pigment Red 185, Pigment Red 208, Pigment Violet 32, Pigment Orange 36, Pigment Orange 62, Pigment Orange 72 or Pigment Brown 25; isoindolinone and isoindoline pigments, in particular the Colour Index pigments Pigment Yellow 139 or Pigment Yellow 173; phthalocyanine pigments, in particular the Colour Index pigments Pigment Blue 15, Pigment Blue 15:1, Pigment Blue 15:2, Pigment Blue 15:3, Pigment Blue 15:4, Pigment Blue 16, Pigment Green 7 or Pigment Green 36; anthanthrone, anthraquinone, quinacridone, dioxazine, indanthrone, perylene, perinone and thioindigo pigments, in particular the Colour Index pigments Pigment Yellow 196, Pigment Red 122, Pigment Red 149, Pigment Red 168, Pigment Red 177, Pigment Red 179, Pigment Red 181, Pigment Red 207, Pigment Red 209, Pigment Red 263, Pigment Blue 60, Pigment Violet 19, Pigment Violet 23 or Pigment Orange 43; triarylcarbonium pigments, in particular the Colour Index pigments Pigment Red 169, Pigment Blue 56 or Pigment Blue 61; diketopyrrolopyrrole pigments, in particular the Colour Index pigments Pigment Red 254.

The pigment is present in the liquid medium in the form of finely divided particles either alone or dispersant stabilized. The average particle diameter is preferably in the range from 1 to 1000 nm and more preferably in the range from 10 to 100 nm. Pigments which can be stabilized without dispersants are known as self-dispersible pigments. These are. normally surface-modified pigments whose surface has been altered by chemical operations such as for example sulfonation or diazotization and has been provided with functional, neutral or charged, groups or polymeric chains (these surface-modified pigments also being known as self-dispersing or graft pigments).

Nonionic, amphoteric, cationic or anionic surfactants or polymers are usable as dispersants.

The present invention also provides aqueous colorant preparations, preferably recording fluids, in particular ink jet inks, comprising 0.1 % to 50% by weight and preferably 0.5% to 20% by weight of at least one heavy metal complex colorant, 0.001% to 0.1% by weight and preferably 0.01% to 0.05% by weight of the antimicrobial mixture in question and 10% to 90% by weight and preferably 30% to 80% by weight of deionized water, all based on the total weight (100% by weight) of the colorant preparation.

The present invention also provides aqueous colorant preparations, preferably recording fluids, in particular ink jet inks, comprising at least 20 ppm of heavy metal ions and 0.1 % to 50% by weight and preferably 0.5% to 20% by weight of at least one colorant, 0.001% to 0.1% by weight and preferably 0.01% to 0.05% by weight of the antimicrobial mixture in question and 10% to 90% by weight and preferably 30% to 80% by weight of deionized water, all based on the total weight (1 00% by weight) of the colorant preparation.

Water used to produce the recording fluids is preferably employed in the form of distilled or demineralized water.

The colorant preparations of the present invention may further comprise, depending on the dyes used, a shading colorant, preferably from the group consisting of C.I. Acid Yellow 17 and C.I. Acid Yellow 23; C.I. Direct Yellow 86, C.I. Direct Yellow 98 and C.I. Direct Yellow 132; C.I. Reactive Yellow 37; C.I. Pigment Yellow 17, C.I. Pigment Yellow 74, C.I. Pigment Yellow 83, C.I. Pigment Yellow 97, C.I. Pigment Yellow 120, C.I. Pigment Yellow 139, C.I. Pigment Yellow 151, C.I. Pigment Yellow 155 and C.I. Pigment Yellow 180; C.I. Direct Red 1, C.I. Direct Red 11, C.I. Direct Red 37, C.I. Direct Red 62, C.I. Direct Red 75, C.I. Direct Red 81, C.I. Direct Red 87, C.I. Direct Red 89, C.I. Direct Red 95 and C.I. Direct Red 227; C.I. Acid Red 1, C.I. Acid Red 8, C.I. Acid Red 80, C.I. Acid Red 81, C.I. Acid Red 82, C.I. Acid Red 87, C.I. Acid Red 94, C.I. Acid Red 115, C.I. Acid Red 131, C.I. Acid Red 144, C.I. Acid Red 152, C.I. Acid Red 154, C.I. Acid Red 186, C.I. Acid Red 245, C.I. Acid Red 249 and C.I. Acid Red 289; C.I. Reactive Red 21, C.I. Reactive Red 22, C.I. Reactive Red 23, C.I. Reactive Red 35, C.I. Reactive Red 63, C.I. Reactive Red 106, C.I. Reactive Red 107, C.I. Reactive Red 112, C.I. Reactive Red 113, C.I. Reactive Red 114, C.I. Reactive Red 126, C.I. Reactive Red 127, C.I. Reactive Red 128, C.I. Reactive Red 129, C.I. Reactive Red 130, C.I. Reactive Red 131, C.I. Reactive Red 137, C.I. Reactive Red 160, C.I. Reactive Red 161, C.I. Reactive Red 174 and C.I. Reactive Red 180; C.I. Pigment Red 122, C.I. Pigment Red 176, C.I. Pigment Red 184, C.I. Pigment Red 185 and C.I. Pigment Red 269; C.I. Direct Blue 199; C.I. Acid Blue 9; C.I. Pigment Blue 15:1-15:4. The shading colorant is preferably present in an amount of 0.001% to 5% by weight and in particular 0.01% to 1% by weight, based on the dry weight of total colorants.

The colorant preparations of the present invention may further comprise organic solvents, humectants, organic or inorganic bases or acids, cationic, anionic or nonionic surface-active substances (surfactants and wetting agents) and also agents for regulating the viscosity, for example polyvinyl alcohol, cellulose derivatives or water-soluble natural or artificial resins as film-formers or binders to enhance the adhesion and abrasion resistance and also photostabilizers in customary amounts.

Examples of suitable humectants are formamide, urea, tetramethylurea, ε-caprolactam, ethylene glycol, propylene glycol, diethylene glycol, triethylene glycol, polyethylene glycol, butylglycol, methylcellosolve, glycerol, N-methylpyrrolidone, 1,3-diethyl-2-imidazolidinone, thiodiglycol, sodium benzenesulfonate, sodium toluenesulfonate, sodium xylenesulfonate, sodium cumenesulfonate, sodium dodecylsulfonate, sodium benzoate, sodium salicylate and sodium butylmonoglycol sulfate.

Examples of suitable solvents are mono- or polyhydric alcohols, their ethers and esters, examples being methanol, ethanol, propanol, isopropanol, butanol, isobutanol; di- or trihydric alcohols, in particular having 2 to 6 carbon atoms, examples being ethylene glycol, propylene glycol, 1,3-propanediol, 1,4-butanediol, 1,5-pentanediol, 1,6-hexanediol, neopentylglycol, 1,2,6-hexanetriol, glycerol, 2-ethyl-2-hydroxymethyl-1,3-propanediol, diethylene glycol, dipropylene glycol, triethylene glycol, polyethylene glycol, tripropylene glycol, polypropylene glycol; lower alkyl ethers of polyhydric alcohols, examples being ethylene glycol monomethyl ether, ethylene glycol monoethyl ether, ethylene glycol monobutyl ether, triethylene glycol monomethyl ether, triethylene glycol monoethyl ether; ketones and ketone alcohols such as for example acetone, methyl ethyl ketone, diethyl ketone, methyl isobutyl ketone, methyl pentyl ketone, cyclopentanone, cyclohexanone, diacetone alcohol; amides, for example dimethylformamide, dimethylacetamide, N-methylpyrrolidone; also urea, tetramethylurea, thiodiglycol, ε-caprolactam.

Examples of suitable bases are ethanolamine, diethanolamine, triethanolamine, N,N-dimethylethanolamine, diisopropylamine.

The recording fluids for the ink jet printing process may comprise still further additives depending on the embodiment of this printing process, for example as continuous jet, intermittent jet, pulsed jet or compound jet process, for example to buffer the pH, to adjust the electrical conductivity, the specific heat, the thermal expansion coefficient and the conductivity.

The colorant preparations of the present invention can be produced by intermixing the stated components in the form of dry powders, their solutions, water- or solvent-moist presscakes.

For use in recording fluids, the colorants described are worked up in accordance with the stated requirements. Dyes may be isolated from the as-synthesized, preferably aqueous, mixtures by salting out and filtering or by spray drying, if appropriate after partial or complete desalting by means of membrane filtration. However, an isolating step may also be dispensed with and the dye-containing reaction mixtures be converted directly into concentrated dye solutions by addition of organic and/or inorganic bases, possibly humectants, preservatives and if appropriate after partial or complete desalting by means of membrane filtration. Alternatively, the dyes can also be employed as a presscake (if appropriate even in flushing processes) or as a powder. Advantageously, the dye mixtures of the present invention are used in an ideally salt-free form, i.e., free of NaCl or other customary inorganic salts formed in the course of the synthesis of the dyes.

In the case of pigment colorants, there is a preferred process wherein, in a first step, at least one pigment (either as a powder or as a presscake) is pasted up in deionized water, if appropriate together with at least one dispersant, if appropriate with at least one organic solvent, if appropriate with at least one hydrotropic substance and if appropriate the other additives, and subsequently homogenized and predispersed using a dissolver or some other suitable apparatus. The subsequent fine dispersion is effected using a bead mill or some other suitable dispersing assembly, the fine dispersion or grinding being effected with cooling to the desired colorant, in particular pigment, particle size distribution. Following fine dispersion, the dispersion can be further diluted with deionized water.

The colorant preparations of the present invention can be used for dyeing and printing natural and synthetic fibrous materials (examples being polyester, silk, wool, blend fabric), in particular for recording script and images on various recording media, and also for dyeing paper or celluloses in the pulp. When recording fluids according to the present invention are stored, there is no separation of precipitates leading to fuzzy printed images or to nozzle clogging. The recording fluids of the present invention are in the ranges suitable for ink jet processes with regard to viscosity and surface tension. They provide printed images of high optical density having excellent light and water fastness. The colorant preparations according to the invention are also useful as colorants in electrophotographic toners and developers, for example one component and two component powder toners or developers, magnetic toners, liquid toners, latex toners, polymerization toners and also other specialty toners. Typical toner binders are addition polymerization, polyaddition and polycondensation resins, e.g., styrene, styrene-acrylate, styrene-butadiene, acrylate, polyester or phenolic epoxy resins, polysulfones and polyurethanes, individually or in combination, and also polyethylene and polypropylene, which may include yet further ingredients, such as charge control agents, waxes or flow agents, or may have added to them subsequently.

The colorant preparations according to the invention are further useful as colorants in powders and powder coatings, especially in triboelectrically or electrokinetically sprayable powder coatings which are used for surface coating articles made for example of metal, wood, plastic, glass, ceramic, concrete, textile material, paper or rubber. Useful powder coating resins typically include epoxy resins, carboxyl- and hydroxyl-containing polyester resins, polyurethanes and acrylic resins together with customary hardeners. Combinations of resins are also used. For instance, epoxy resins are frequently used in combination with carboxyl- and hydroxyl-containing polyester resins. Typical hardener components (depending on the resin system) are for example acid anhydrides, imidazoles and also dicyandiamide and their derivatives, capped isocyanates, bisacylurethanes, phenolic and melamine resins, triglycidyl isocyanurates, oxazolines and dicarboxylic acids.

The colorant preparations according to the invention are also useful as colorants for color filters and also for additive as well as subtractive color generation and also as colorants for electronic inks (“e-inks”) or electronic paper (“e-paper”).

The antimicrobial mixture used according to the present invention possesses high effectiveness against a multiplicity of customary germs over a wide pH range. There are no precipitates of heavy metal-active complexes even on prolonged storage.

In the examples which follow, parts are by weight.

### EXAMPLE 1

1000 parts of a 10% by weight aqueous solution of Direct Blue 199 (5a) (as-synthesized content of ionic copper: 80 ppm) are admixed with various biocides to the concentrations reported in Table 1. An additional 100 ppm of divalent cations (in the form of their metal salts) is added in the other tests. After stirring, the resulting solutions are suction filtered through a paper filter (Schleicher&Schuell Ref. No. 14812). The paper filter is inspected for the appearance of a precipitate due to the formation of the corresponding biocide-heavy metal complex. The results are reported in Table 1.

TABLE 1
(10% by weight solution of Direct Blue 199)

| | | Pure dye | | | |
|---|---|---|---|---|---|
| Active ingredient | Trade name | solution | Cu²⁺ | Zn²⁺ | Fe³⁺ |
| 0.01% of BIT | Mergal K10N | + | ++ | ++ | ++ |
| | (Troy) | | | | |
| 0.01% of BIT | Nipacide BIT20 | + | ++ | ++ | ++ |
| | (Clariant) | | | | |
| 0.01% of BIT | Proxel GXL | + | ++ | ++ | ++ |
| | (Avecia) | | | | |
| 0.018% of bronopol | Nipacide IB | — | — | — | — |
| 0.0011% of CMIT | (Clariant) | | | | |
| 0.00037% of MIT | | | | | |

— no precipitate
+ formation of a precipitate
++ severe formation of a precipitate
BIT 1,2-benzisothiazolin-3-one

It emerges that the biocide mixture of the present invention, unlike conventional biocides, does not form insoluble complexes with any of the heavy metal cations investigated. The comparative biocides lead to a severe formation of precipitates in the presence of Cu²⁺, Zn²⁺ or Fe³⁺.

### EXAMPLE 2

1000 parts of a 10% by weight aqueous solution of Direct Blue 199 (5a) are mixed with 0.18 part of 2-bromo-2-nitropropane-1,3-diol, 0.011 part of 5-chloro-2-methyl-4-isothiazolin-3-one and 0.0037 part of 2-methyl-4-isothiazolin-3-one and also 0.018 part of magnesium nitrate and 0.01 part of magnesium chloride.

### EXAMPLE 3

1000 parts of a 15% by weight aqueous solution of Reactive Black 31 (5b) are mixed with 0.18 part of 2-bromo-2-nitropropane-1 ,3-diol, 0.011 part of 5-chloro-2-methyl-4-isothiazolin-3-one and 0.0037 part of 2-methyl-4-isothiazolin-3-one and also 0.018 part of magnesium nitrate and 0.01 part of magnesium chloride. The two dye solutions of Direct Blue 199 and Reactive Black 31 and also the corresponding control dye solutions without biocides were subjected to a challenge test by admixing them on a weekly basis with a microorganism mixture of the following bacteria, fungi and yeasts:

bacteria:

Pseudomonas versicolor

Alcaligenens faecalis

Enterobacter aerogenes

Escherichia coli

Providencia rettgeri

Myroides odoratus

fungi:

Fusarium solani

Geotrichum candidum

Aspergillus terreus

yeast:

Rhodotorula rubra

The germ load was subsequently tested by determining the number of germs on suitable nutrient media. Severe germ growth was detected after just a few days in the case of the two control samples without biocide, whereas no viable microorganisms could be detected in the case of the dye solution comprising biocide mixture even after 5 weeks.

### EXAMPLES 4 to 7

### Pigments

The pigment, either as a powder or as a presscake, was pasted up in deionized water together with the dispersants, the organic solvent and the other additives and then homogenized and predispersed using a dissolver. The subsequent fine dispersion was effected using a bead mill, the grinding being effected with cooling to the desired pigment particle size distribution. Subsequently, the dispersion was adjusted to the desired final pigment concentration with deionized water.

The pigment preparations described in the Examples 4 to. 7 hereinbelow were produced by the process described above, the following constituents being used in the stated amounts such that 100 parts of the respective pigment preparation were produced:

### EXAMPLE 4

| | | |
|---|---|---|
| 20 | parts | of Pigment Yellow 155 |
| 2.5 | parts | of acrylate resin, sodium salt (dispersant) |
| 1.2 | parts | of polyethylene glycol alkyl ether, sodium salt |
| | | (dispersant) |
| 0.018 | part | of 2-bromo-2-nitropropane-1,3-diol |
| 0.0011 | part | of 5-chloro-2-methyl-4-isothiazolin-3-one |
| 0.00037 | part | of 2-methyt-4-isothiazolin-3-one |
| 0.0018 | part | of magnesium nitrate |
| 0.001 | part | of magnesium chloride |

| | |
|---|---|
| Balance | deionized water |

### EXAMPLE 5

| | | |
|---|---|---|
| 20 | parts | of Pigment Red 122 |
| 2.5 | parts | of acrylate resin, sodium salt (dispersant) |
| 1.2 | parts | of polyethylene glycol alkyl ether, sodium salt |
| | | (dispersant) |
| 0.018 | part | of 2-bromo-2-nitropropane-1,3-diol |
| 0.0011 | part | of 5-chloro-2-methyl-4-isothiazolin-3-one |
| 0.00037 | part | of 2-methyl-4-isothiazolin-3-one |
| 0.0018 | part | of magnesium nitrate |
| 0.001 | part | of magnesium chloride |

| | |
|---|---|
| Balance | deionized water |

### EXAMPLE 6

| | | |
|---|---|---|
| 20 | parts | of Pigment Red 15:3 |
| 2.5 | parts | of acrylate resin, sodium salt (dispersant) |
| 1.2 | parts | of polyethylene glycol alkyl ether, sodium salt |
| | | (dispersant) |
| 0.018 | part | of 2-bromo-2-nitropropane-1,3-diol |
| 0.0011 | part | of 5-chloro-2-methyl-4-isothiazolin-3-one |
| 0.00037 | part | of 2-methyl-4-isothiazolin-3-one |
| 0.0018 | part | of magnesium nitrate |
| 0.001 | part | of magnesium chloride |

| | |
|---|---|
| Balance | deionized water |

### EXAMPLE 7

| | | |
|---|---|---|
| 15 | parts | of carbon black (Pigment Black 7) |
| 2.5 | parts | of acrylate resin, sodium salt (dispersant) |
| 0.6 | part | of polyethylene glycol alkyl ether, sodium salt |
| | | (dispersant) |
| 0.018 | part | of 2-bromo-2-nitropropane-1,3-diol |
| 0.0011 | part | of 5-chloro-2-methyt-4-isothiazolin-3-one |
| 0.00037 | part | of 2-methyl-4-isothiazolin-3-one |
| 0.0018 | part | of magnesium nitrate |
| 0.001 | part | of magnesium chloride |

| | |
|---|---|
| Balance | deionized water |

### COMPARATIVE EXAMPLE

Example 4 was repeated with the biocide omitted, resulting in the following composition:

| | | |
|---|---|---|
| 20 | parts | of Pigment Yellow 155 |
| 2.5 | parts | of acrylate resin, sodium salt (dispersant) |
| 1.2 | parts | of polyethylene glycol alkyl ether, sodium salt (dispersant) |

| | |
|---|---|
| Balance | deionized water |

The germ load was subsequently tested by determining the number of germs on suitable nutrient media. Severe germ growth was detected after just a few days in the case of the control sample without biocide (comparative example), whereas viable microorganisms could not be detected in the case of the pigment dispersions comprising biocide mixture (Examples 4-7) even after 1 week.

1) A method for increasing the antimicrobial effect of an aqueous colorant preparation having a heavy metal ion concentration of not less than 20 ppm and wherein the colorant is a reactive dye, a direct dye, an acid dye, a disperse dye or a pigment and includes a complexed heavy metal selected from the group consisting of Cu, Co, Ni, Fe, Cr and Al comprising the step of adding to the agueous colorant an effective amount of a biocide. wherein the biocide is an antimicrobially effective amount of a mixture of 5-chloro-2-methyl-4-isothiazolin-3-one, 2-methyl-4-isothiazolin-3-one and 2-bromo-2-nitropropane-1,3-diol.

2) The method according to claim 1 wherein the aqueous colorant preparation is a recording fluid.

3) The method according to claim 1 wherein the antimicrobially effective amount is 0.001% to 0.1% by weight and as solid and based on the total weight of the colorant preparation.

4) The method according to claim 1 wherein the mixing ratio of the individual constituents chloro-2-methyl-4-isothiazolin-3-one, 2-methyl-4-isothiazolin-3-one and 2-bromo-2-nitropropane-1,3-diol is (0.005 to 0.1):(0.005 to 0.05):1.

5) (canceled)

6) The method according to claim 1 wherein the colorant is C.I. Reactive Black 8, C.I. Reactive Black 31, C.I. Reactive Blue 7, C.I. Reactive Blue 14, C.I. Reactive Blue 21, C.I. Reactive Blue 28, C.I. Reactive Blue 38, C.I. Reactive Blue 82, C.I. Reactive Blue 89, C.I. Reactive Blue 158, C.I. Reactive Blue 182, C.I. Reactive Blue 190, C.I. Reactive Blue 203, C.I. Reactive Blue 216, C.I. Reactive Blue 220, C.I. Reactive Blue 244, C.I. Reactive Violet 1, C.I. Reactive Violet 5, C.I. Reactive Red 6, C.I. Reactive Red 23, C.I. Reactive Brown 18, C.l Direct Blue 76, C.I. Direct Blue 84, C.I. Direct Blue 86, C.I. Direct Blue 87, C.I. Direct Blue 98, C.I. Direct Blue 199, C.I. Direct Blue 202, C.I. Direct Blue 290, C.I. Direct Black 112, C.I. Direct Brown 95, C.I. Direct Violet 47, C.I. Acid Blue 87, C.I. Acid Blue 185, C.I. Acid Blue 249, C.I. Pigment Blue 15:1-15:4, C.I. Pigment Blue 17, C.I. Pigment Green 7, C.I. Pigment Green 37, C.I. Pigment Red 257, C.I. Pigment Red 271, C.I. Pigment Orange 65, C.I. Pigment Orange 68, C.I. Pigment Yellow 117, C.I. Pigment Yellow 129 or C.I. Pigment Yellow 153.

7) An aqueous colorant preparation comprising 0.1 % to 50% by weight of at least one heavy metal complex colorant, 0.001% to 0.1% by weight of an antimicrobial mixture, wherein the antimicrobial mixture is a mixture of 5-chloro-2-methyl-4-isothiazolin-3-one. 2-methyl-4-isothiazolin-3-one and 2-bromo-2-nitropropane-1,3-diol according to one or more of claims 1 to 4 and 10% to 90% by weight of deionized water, all based on the total weight (100% by weight) of the colorant preparation.

8) An aqueous colorant preparation comprising at least 20 ppm of heavy metal ions and 0.1 % to 50% by weight of at least one colorant 0.001 % to 0.1 % by weight of an antimicrobial mixture wherein the antimicrobial mixture is a mixture of 5-chloro-2-methyl-4-isothiazolin-3-one, 2-methyl-4-isothiazolin-3-one and 2-bromo-2-nitropropane-1.3-diol and 10% to 90% by weight of deionized water, all based on the total weight (100% by weight) of the colorant preparation.

9) The aqueous colorant preparation according to claim 7 further comprising a shading colorant selected from the group consisting of C.I. Acid Yellow 17 and C.I. Acid Yellow 23; C.I. Direct Yellow 86, C.I. Direct Yellow 98 and C.I. Direct Yellow 132; C.I. Reactive Yellow 37; C.I. Pigment Yellow 17, C.I. Pigment Yellow 74, C.I. Pigment Yellow 83, C.I. Pigment Yellow 97, Cl. Pigment Yellow 120, Cl. Pigment Yellow 139, Cl. Pigment Yellow 151, C.I. Pigment Yellow 155 and C.I. Pigment Yellow 180; C.I. Direct Red 1, C.I. Direct Red 11, C.I. Direct Red 37, C.I. Direct Red 62, C.I. Direct Red 75, C.I. Direct Red 81, C.I. Direct Red 87, C.I. Direct Red 89, C.I. Direct Red 95 and C.I. Direct Red 227; C.I. Acid Red 1, C.I. Acid Red 8, C.I. Acid Red 80, C.I. Acid Red 81, C.I. Acid Red 82, C.I. Acid Red 87, C.I. Acid Red 94, C.I. Acid Red 115, C.I. Acid Red 131, C.I. Acid Red 144, C.I. Acid Red 152, C.I. Acid Red 154, C.I. Acid Red 186, C.I. Acid Red 245, C.I. Acid Red 249 and C.I. Acid Red 289; C.I. Reactive Red 21, C.I. Reactive Red 22, C.I. Reactive Red 23, C.I. Reactive Red 35, C.I. Reactive Red 63, C.I. Reactive Red 106, C.I. Reactive Red 107, C.I. Reactive Red 112, C.I. Reactive Red 113, C.I. Reactive Red 114, C.I. Reactive Red 126, C.I. Reactive Red 127, C.I. Reactive Red 128, C.I. Reactive Red 129, C.I. Reactive Red 130, C.I. Reactive Red 131, C.I. Reactive Red 137, C.I. Reactive Red 160, C.I. Reactive Red 161, C.I. Reactive Red 174 and C.I. Reactive Red 180; C.I. Pigment Red 122, C.I. Pigment Red 176, C.I. Pigment Red 184, C.I. Pigment Red 185 and C.I. Pigment Red 269; C.I. Direct Blue 199; C.I. Acid Blue 9 and C.I. Pigment Blue 15:1-15:4 in an amount of 0.001 % to 5% by weight and in particular of 0.01 % to 1 % by weight, based on the dry weight of the entire colorant.

10. The method according to claim 1, wherein the recording fluid is a ink jet ink.

11. The method according to claim 1 wherein the antimicrobially effective amount 0.02% by weight, as solid and based on the total weight of the colorant preparation.

12. The aqueous colorant composition as claimed in claim 9, wherein the shading colorant is present in an amount of from 0.01 % to 1% by weight.

13. A composition produced with the aqueous colorant composition as claimed in claim 7, wherein the composition is selected from the group consisting of powders and powder coatings, triboelectrically powder coatings and electrokinetically sprayable powder coatings.

14. A composition produced with the aqueous colorant composition as claimed in claim 7, wherein the composition is selected from the group consisting of color filters, electronic inks and electronic paper.

15. A fiberous composition produced with the aqueous colorant composition as claimed in claim 7, wherein fiberous composition is a natural or synthetic material.
